(19) United States
(12) Patent Application Publication
(10) Pub. No.: US 2008/0044455 A1
(43) Pub. Date: Feb. 21, 2008

(54) Tonsillitus Treatment

(76) Inventors: Chaim Welczer, Palo Alto, CA (US); Avelyn Welczer, Palo Alto, CA (US)

Correspondence Address: PATTERSON & SHERIDAN, L.L.P., 3040 POST OAK BOULEVARD, SUITE 1500, HOUSTON, TX, 77056, US

(21) Appl. No.: 11/835,112

(22) Filed: Aug. 7, 2007

Related U.S. Application Data
(60) Provisional application No. 60/823,074, filed on Aug. 21, 2006.

(51) Int. Cl. A61K 31/185; A61K 47/00; A61K 9/12; A61K 9/20; A61P 11/04

(52) U.S. Cl. 424/439; 424/43; 424/464; 514/578

(57) ABSTRACT

A therapeutic agent and methodology for treatment of tonsillitis and tonsil, throat and mouth infection is provided as a combination of Lactic Acid and a delivery vehicle, such as an ingestible liquid. The agent is ingested by an individual with a spacing between applications thereof, such that recurrence of tonsil infection is suppressed.

### CROSS-REFERENCE TO RELATED APPLICATIONS

This application claims benefit of U.S. provisional patent application Ser. No. 60/823,074, filed Aug. 21, 2006, which is herein incorporated by reference.

The present invention relates to the field of therapeutic remedies, more specifically to the field of remedies for throat inflammation and infection.

A common approach for the treatment of throat inflammation, as well as for the treatment of tonsillitis, focuses on systemic use of antibiotics. Antibiotics such as penicillin, were once effectively used to eradicate bacterial infections, which caused throat or tonsil inflammation. Over time, and with widespread use of antibiotics, the efficacy of such antibiotics diminished as the infectious agents become genetically modified, and thus became resistant to prescribed antibiotics. As such, the treatment of infection has become more difficult, and has required antibiotics of greater strength, and having additional or greater side effects. This paradigm has created a vicious cycle, wherein new antibiotics are invented or discovered which will treat the infection, but those genetic modifications or variants of the infective bacteria, which are immune to, or can, survive treatment by, the new antibiotics survive and multiply, creating a new, antibiotic resistant, variant of the infectious agent. One cause of this paradigm is the over-prescribing of antibiotics, such that antibiotics are used to treat infection where other treatments may be as, or nearly as, effective as the treatment with an antibiotic, or where antibiotics are prescribed as a placebo for an underlying viral infection. Thus, there is a desire in the body of medical practitioners to reduce the prescribing of, and the duration of treatment with antibiotics, so as to reduce the likelihood of developing further antibiotic-resistant mutations of infectious agents.

Where tonsillitis persists in the face of antibiotic treatment, a tonsillectomy, e.g., the removal of the tonsils, is prescribed. This surgical procedure typically requires anesthesia and a hospital stay for recovery, which are highly traumatic for the likely individual undergoing the tonsillectomy: A child, adolescent or adult.

Thus, there is a need in the art to provide a non-antibiotic based treatment for tonsillitis, such that the use of antibiotics is reduced or eliminated and the incidence of surgical intervention to remove the tonsils is reduced or eliminated.

### SUMMARY OF THE INVENTION

Application of C₃H₆O₃, commonly known as Lactic Acid, is topically provided to the inflammation of the throat, back of the mouth, and or tonsils, to reduce infection. It has been found that such topical application of lactic acid, such as by the dilution of lactic acid in a liquid carrier, such as a juice and the drinking or otherwise topically applying such a mixture to the tonsils, throat and back of the mouth, results in the reduction of both inflammation and pain in the treated areas, and in a reduction in the recurrence of the inflammation and infection causing the inflammation due to the reduction of the size of the tonsils; there by increasing the distance of the tonsils from each other and adjacent mouth and throat tissue thereby leaving less confined space for bacteria to grow.

In one aspect, a mixture of 10% by volume of pharmaceutical lactic acid is mixed with 90% by volume liquid carrier, such as juice, and the mixture is ingested 3 times a day for a month at a dosage of one teaspoon (approximately 0.005 liters) per application. After a two week pause, the patient is evaluated for the reduction in size of the tonsils. If inadequate reduction in the size of the tonsils, i.e., if they have not shrunk such that a large gap occurs between them, the treatment is continued for another month. The number of applications per day can be reduced to two times a day, in the event that it is hard to implement with the patient, thus extending the duration of the treatment by 10 days.

In another aspect, first ingestion occurs at a prescribed date, and the second ingestion some time thereafter. The second administration (of a plurality of doses or ingestions over time) may in one aspect occur 40 to 45 days after the first administration. The first administration may be responsive to an indication that throat inflammation or tonsillitis is present, or may be prophylactic. The first administration may, in another aspect, be administered at an age of about 3, depending on the child's health and frequency of inflammations that are very often accompanied with ear infections.

### DESCRIPTION OF THE DRAWING

FIG. 1 is a flowchart setting forth the preparation and application of the treatment for the reduction in tonsil size.

### DESCRIPTION OF THE EMBODIMENTS

A method and an article useful for practicing the method are provided for the treatment of throat and tonsil inflammation. In one aspect, the article is a mixture of Lactic Acid and a carrier, such as a liquid, and the method includes ingesting such as by drinking the mixture, which as it is ingested contacts the membranes or surfaces of the throat and tonsils.

In another aspect, the article, i.e., the mixture of Lactic Acid and carrier, to be ingested can be in the form of a frozen popsicle, or other methods of delivering the article such as room temperature solid popsicle, or “candy” that dissolves in the mouth when in contact with the saliva, and then contacts the membranes or surfaces of the throat and tonsils.

In another aspect, the article, i.e., the mixture of Lactic Acid and carrier to be ingested can be in the form of a chewing gum that releases the active component when in contact with the saliva as it is being chewed, and then contacts the membranes or surfaces of the throat and tonsils.

In another aspect, the article to be ingested is made using other acid than Lactic Acid as long as it is in concentrations that do not create a burn or toxic situation in the human body.

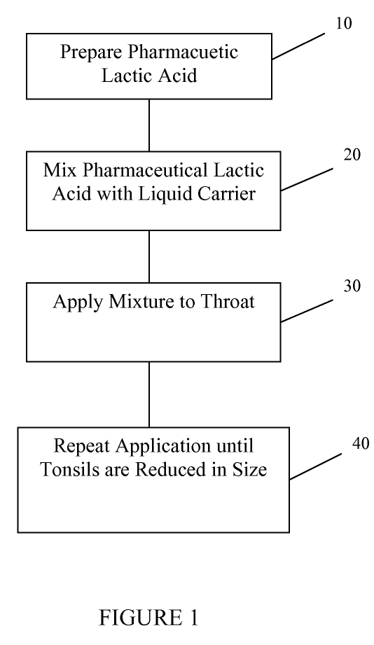

Referring now to FIG. 1, to prepare the article, pharmaceutical grade lactic acid is obtained or formulated as at step 10, and this is then combined, with a volume of a carrier liquid as in step 20, in one aspect a berry juice, with pharmaceutical grade lactic acid as at step 30, to provide an ingestible, by oral intake, solution or mixture which may be ingested by the drinking thereof as at step 40, such that the lactic acid is contacted with the tonsils, throat and back of the mouth for therapeutic effect. In one aspect, the mixture or solution is ingested, such as by repeatedly drinking, as at step 50, over a period of time such as a week, to topically apply the mixture or solution constituents to the surfaces of the tonsils, throat and/or mouth, at least twice, with a duration of time between the two applications. It has been found that by diluting Lactic Acid in a fruit juice, such as raspberry juice, and topically applying the juice-Lactic Acid mixture to the throat, tonsils and mouth, the incidence of tonsillitis is reduced as the tonsils are athrophiated, i.e., diminished in size.

The mixture of Lactic Acid and juice preferably includes one part lactic acid to 9 parts juice, although ranges on the order of 0.01% to 60% of Lactic Acid to total Lactic acid-juice volume are contemplated. Additionally, a range of 0.01% to 60% lactic acid to total volume is likewise envisioned, with a range of 1% to 15% being preferred.

Juice such as raspberry juice, which is pleasant to the palate of children, adolescents and adults and also has an extended shelf life, has been found to be an acceptable dilutent or juice for the mixture. However, any fruit juice or other carrier which has an extended shelf life, but likewise does not negatively interact with the Lactic Acid, is specifically contemplated. Thus, juices, such as grape juice, apple juice, strawberry juice, etc., are specifically contemplated.

The Juice-Lactic Acid mixture may be prepared locally for immediate use, or may be prepared for bottling, in the case of liquid, or solid packaging, in the case of room temperature popsicles; and distribution. In either case, a method of application thereof includes the following steps:
Ingestion of the mixture so as to coat or contact the tonsils, throat and mouth with the mixture; and thereafter;
Ingestion of the mixture so as to coat or contact the tonsils, throat and mouth with the mixture at a time period after the first ingestion.

Each ingestion may include a number of small doses in a particular time, such as two or three teaspoon sized (approximately five cubic centimeters) volume of the mixture daily for five to seven days. The mixture may be prepared once, and stored, or prepared immediately before each administration. Additionally, the concentration of the lactic acid in the carrier may remain the same at each administration, or if desired, varied.

Although the invention is described in terms of ingestion of a liquid mixture such as by the drinking thereof, and the mixture comprising a fruit juice and Lactic Acid, other variants are specifically contemplated. In one variant, a liquid other than a fruit juice is mixed with the Lactic Acid in the aforesaid percentages of total non-Lactic Acid volume percentage. These additional liquids may form the entire volume of non-Lactic Acid liquid, or may be combined with the afore said fruit juice.

In another aspect, mechanisms other than ingestion by drinking of a liquid-Lactic Acid mixture may be used to contact the liquid-Lactic Acid mixture with the tonsils, back of the mouth and throat. In one aspect, this includes forming a lozenge having Lactic Acid therein in a similar concentration of Lactic Acid to solid volume as was present with the liquid-Lactic Acid mixture, such that an individual may suck on or otherwise allow the lozenge to dissolve in the mouth and thereby release a lactic acid-mixture which can contact the tonsils, back of mouth and throat for therapeutic effect. Likewise effervescent strips having the mixture in releasable form maybe used. Again, as with the liquid-Lactic Acid mixture, at least two applications, each with multiple dosings over a period of time, are specifically contemplated.

In yet another aspect, a vapor or liquid droplets are combined with Lactic Acid in a mixture having the same relative percentages of Lactic Acid to remaining mixture components as was present with the liquid-Lactic Acid mixture. The liquid droplets, spray or Lactic Acid mixture may be delivered in an aerosol application, such that the mixture is held under pressure in a container, such that a metered dose may be applied by enabling a propellant to aerosolize the mixture and expel it from the container such that the individual taking up the mixture may relatively easily aim the mixture as it exits the container, at the tonsils, throat and back of the mouth. Alternatively, the mixture may be held in a spray type apparatus, wherein a pressure, or a trigger mechanism, is used to expel a relatively uniform quantity of the liquid-Lactic Acid mixture which can be aimed at the tonsils, throat and back of the mouth through an open mouth. Likewise, a vapor of lactic acid, or lactic and a liquid carrier, can be provided by sublimating a solid or vapor arising from liquid, wherein the vapor is inhaled by the patient. Again, multiple applications of the vapor or liquid droplet mixture, with a delay or period between applications is provided.

In each of the described methodologies of an application, as well as the articles used for the methodologies commonly include a first treatment period, over which period, over which period a quantity of the article is applied sequentially to at least the tonsils, after which second, waiting period is provided. After the waiting period, the patients' tonsils are viewed, and their size, as well as the spacing of the tonsils to one another as well as to adjacent mouth tissue is evaluated. This evaluation may be an initial observation, or may be in comparison with an earlier observation. If additional change, ie, reduction of the size of the tonsil is desired, the treatment methodology, including multiple doses over a period of time, is again provided. Additionally, multiple applications on the first and later day of application may be necessary to sufficiently coat the areas to be treated with the mixture.

Although the application of the Lactic Acid via a mixture of Lactic Acid and juice, Lactic Acid and a liquid, and Lactic Acid and an aerosol or droplet have been described, the first application of the Lactic Acid may be by any of the carriers, such as the juice ingested by drinking, the liquid ingested by drinking, the frozen popsicle, the lozenge, the aerosol or the droplet methodology, and the second application being of a second methodology selected from these or other methodologies sufficient to coat the tonsils, back of the mouth and/or the throat with the carrier-Lactic Acid mixture.

I claim:

1. A method of providing therapeutic treatment, comprising:
providing a first quantity of a liquid and a second quantity of Lactic Acid additive to a third quantity;
providing the second quantity such that the relative percentage of the second quantity to the third quantity is between 0.01% to 60%; and
providing the third quantity for oral ingestion.

2. The method of claim 1, further including the step of providing the second quantity such that the relative percentage of the second quantity to the third quantity is on the order of 1% to 15%.

3. The method of claim 1, further including the step of providing the second quantity such that the relative percentage of the second quantity to the third quantity is on the order of 10%.

4. The method of claim 1, further including the steps of:
providing a storage receptacle for third quantity;
providing instructions for oral ingestion of the third quantity; and
providing the third quantity to an individual.

5. The method of claim 1, wherein the step of oral ingestion is by drinking of the mixture.

6. The method of claim 1, wherein the step of oral ingestion includes the steps of:
a. forming the third quantity as a frozen popsicle; and
b. instructing the maintenance of the frozen popsicle in a subject's mouth to release the lactic acid therein.

7. The method of claim 1, wherein the step of oral ingestion includes the steps of:
i) forming the third quantity as a lozenge; and
ii) instructing the maintenance of the lozenge in a subject's mouth to release the lactic acid therein.

8. The method of claim 1, wherein the step of oral ingestion includes the step of directing a vapor or a droplet spray into the mouth.

9. A therapeutic agent, comprising;
a first quantity of an ingestible agent and a second quantity of Lactic Acid additive to a third quantity;
the second quantity having a quantity percentage relative to the quantity of the third quantity of between 0.01% to 60%; and
instruction for the application of the third quantity for oral ingestion.

10. The therapeutic agent of claim 8, wherein said ingestible agent includes a liquid.

11. The therapeutic agent of claim 9, wherein said liquid is a fruit juice.

12. The therapeutic agent of claim 8, wherein said third quantity is in the form of a frozen popsicle.

13. The therapeutic agent of claim 8, wherein said third quantity is in the form of a lozenge.

14. The therapeutic agent of claim 8, wherein said third quantity is provided in aerosol form.

15. The therapeutic agent of claim 8, wherein the therapeutic agent is deliverable for ingestion in droplet form.
